(19) United States
(12) Patent Application Publication
(10) Pub. No.: US 2022/0230076 A1
(43) Pub. Date: Jul. 21, 2022

(54) DATA PROCESSING METHOD AND APPARATUS FOR MACHINE LEARNING

(71) Applicant: FUJITSU LIMITED, Kawasaki-shi (JP)

(72) Inventors: Yoshihiro OKAWA, Yokohama (JP); MASARU IDE, Setagaya (JP)

(73) Assignee: FUJITSU LIMITED, Kawasaki-shi (JP)

(21) Appl. No.: 17/714,555

(22) Filed: Apr. 6, 2022

Related U.S. Application Data
(63) Continuation of application No. PCT/JP2019/041466, filed on Oct. 23, 2019.

(51) Int. Cl. G06N 5/02

(52) U.S. Cl. CPC G06N 5/022

(57) ABSTRACT

A processor generates training data by performing a process, based on a parameter, on first measurement data. The processor trains a machine learning model by using the training data. The processor generates first data by performing the process on second measurement data. The processor generates a first prediction result by entering the first data into the machine learning model, and calculates prediction accuracy based on a label associated with the second measurement data and the first prediction result. The processor changes the parameter of the process in accordance with a comparison between the training data and the first data in response to the predication accuracy being less than a threshold.

### CROSS-REFERENCE TO RELATED APPLICATION

This application is a continuation application of International Application PCT/JP2019/041466 filed on Oct. 23, 2019 which designated the U.S., the entire contents of which are incorporated herein by reference.

### FIELD

The embodiments discussed herein relate to machine learning.

### BACKGROUND

Machine learning may be used for computer-based data analysis. In the machine learning, training data representing known cases is entered into a computer. The computer then analyzes the training data and trains a model to generalize the relationship between causes (called “explanatory variable” or “independent variable”) and results (called “response variable” or “dependent variable”). Using the trained model, the computer is able to predict the results of unknown cases.

A series of data analysis using machine learning may be classified into a training phase of collecting past data and training a model and an application phase of entering data obtained after the training into the model to predict a result. It is noted that as time passes, the data that is entered into the model in the application phase may have a different tendency from that used in the training phase. Due to the change in the tendency, the prediction of the model may become less accurate at a later time. To deal with this, the retraining of the model may be considered as one way to recover the prediction accuracy.

For example, there has been proposed a wind power prediction method of predicting future wind power production using past wind power production and weather prediction. This proposed wind power prediction method trains a model through machine learning and constantly retraining the model using latest data. In addition, there has been proposed a continual machine learning method of continually updating a model to catch up with the trend of input data. This proposed continual machine learning method determines when to update the model with taking into account a tradeoff between a delay to the incorporation of latest data into the model and the cost of machine learning.

Related arts are disclosed in, for example, Mariam Barque, Simon Martin, Jeremie Etienne Norbert Vianin, Dominique Genoud and David Wannier, “Improving wind power prediction with retraining machine learning algorithms”, Proc. of the 2018 International Workshop on Big Data and Information Security (IWBIS 2018), pp. 43-48, 2018-05-12, and Huangshi Tian, Minchen Yu and Wei Wang, “Continuum: A Platform for Cost-Aware, Low-Latency Continual Learning”, Proc. of the ACM Symposium on Cloud Computing 2018 (SoCC'18), pp. 26-40, 2018-10-11.

### SUMMARY

According to one aspect, there is provided a computer-implemented data processing method including: generating training data by performing a process, based on a parameter, on first measurement data; training a machine learning model by using the training data; generating first data by performing the process on second measurement data; generating a first prediction result by entering the first data into the machine learning model, and calculating prediction accuracy based on a label associated with the second measurement data and the first prediction result; and changing the parameter of the process in accordance with a comparison between the training data and the first data in response to the predication accuracy being less than a threshold.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a view for explaining an example of a data preprocessing apparatus according to a first embodiment;

FIG. 2 illustrates an example of hardware configuration of a machine learning apparatus according to a second embodiment;

FIG. 3 illustrates an example of a sequence of training and applying a model;

FIG. 4 illustrates an example of a sequence of decrease due to noise and recovery in prediction accuracy;

FIG. 5 illustrates an example of searching for a parameter of a preprocessing filter;

FIG. 6 illustrates an example of generating training data;

FIG. 7 illustrates an example of abnormality detection by a k-nearest neighbor model;

FIG. 8 illustrates an example of erroneous detection with respect to an input sample with noise;

FIG. 9 illustrates an example of searching for a parameter of a low-pass filter;

FIG. 10 illustrates an example of applying a first low-pass filter;

FIG. 11 illustrates an example of applying a second low-pass filter;

FIG. 12 illustrates an example of applying a third low-pass filter;

FIG. 13 is a block diagram illustrating an example of functions of the machine learning apparatus;

FIG. 14 illustrates an example of a measurement data table;

FIG. 15 illustrates an example of a filter table;

FIG. 16 is a flowchart illustrating an example of a training stage process; and

FIG. 17 is a flowchart illustrating an example of an application stage process.

### DESCRIPTION OF EMBODIMENTS

Data that is used in machine learning may be measurement data that is obtained by a measurement device, such as time-series signal data or image data. Such measurement data may contain noise due to the characteristics and usage environment of the measurement device. Therefore, a change in the tendency of noise may occur as one of changes in the tendency of data. For example, a noise pattern that does not occur in the training phase may appear in the measurement data due to the aging of the measurement device and a change in the usage environment thereof. However, to retrain a model every time the tendency of data changes costs a lot in terms of computational complexity and training time.

Some embodiments will be described below with reference to the accompanying drawings.

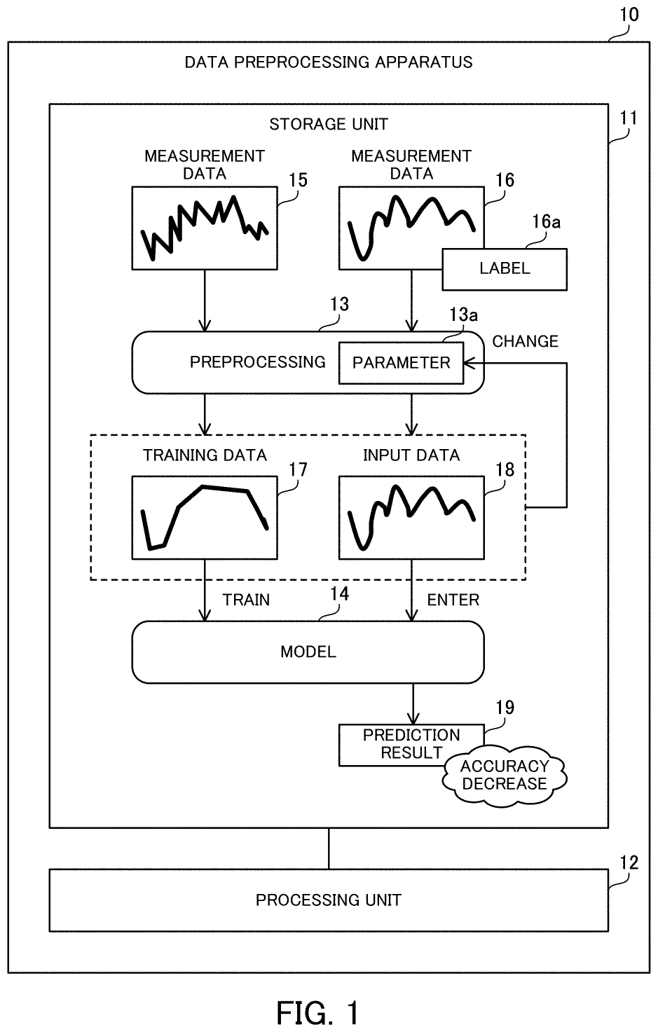

FIG. 1 is a view for explaining an example of a data preprocessing apparatus according to the first embodiment.

The data preprocessing apparatus 10 of the first embodiment trains a model through machine learning and predicts a result corresponding to input data using the trained model. Training data that is used for training the model and input data that is entered into the model go through preprocessing. The data preprocessing apparatus 10 may be a client apparatus or a server apparatus. The data preprocessing apparatus 10 may be called a computer, an information processing apparatus, a machine learning apparatus, or another. In the first embodiment, the data preprocessing apparatus 10 executes both a training phase of training a model and an application phase of using the model. Alternatively, these phases may be executed by different apparatuses.

The data preprocessing apparatus 10 includes a storage unit 11 and a processing unit 12. The storage unit 11 may be a volatile semiconductor memory device, such as a random access memory (RAM), or a non-volatile storage device, such as a hard disk drive (HDD) or a flash memory. The processing unit 12 is a processor such as a central processing unit (CPU), a graphics processing unit (GPU), or a digital signal processor (DSP), for example. In this connection, the processing unit 12 may include an application specific integrated circuit (ASIC), a field programmable gate array (FPGA), or another application specific electronic circuit. The processor runs programs stored in a memory (this may be the storage unit 11) such as a RAM. A set of multiple processors may be called “a multiprocessor” or simply “a processor.”

The storage unit 11 holds a parameter 13a, a model 14, measurement data 15 (first measurement data), measurement data 16 (second measurement data), a label 16a associated with the measurement data 16, training data 17, input data 18, and a prediction result 19.

The parameter 13a is a control parameter for controlling the behavior of preprocessing 13. In the following description, the term “parameter” has its ordinary meaning and also includes a parameter value. The preprocessing 13 converts the measurement data 15 into the training data 17 at the time of training the model 14. The preprocessing 13 also converts the measurement data 16 into the input data 18 at the time of using the model 14.

For example, the preprocessing 13 functions as a noise filter to remove noise from the measurement data 15 and 16. The preprocessing 13 may function as a low-pass filter to remove high-frequency components, as a high-pass filter to remove low-frequency components, or as a bandpass filter to remove frequency components other than predetermined frequencies. The parameter 13a may be set to specify a cutoff frequency indicating a boundary for cutting off frequencies. Alternatively, the parameter 13a may be set to specify a coefficient to implement a filter such as a finite impulse response (FIR) filter or an infinite impulse response (IIR) filter.

The model 14 is a machine learning model that generalizes the relationship between an explanatory variable and a response variable. The model 14 is created by a predetermined machine learning algorithm using the training data 17. The trained model 14 accepts an input of the input data 18 corresponding to the explanatory variable and outputs the prediction result 19 corresponding to the response variable. In the first embodiment, any of various machine learning algorithms is usable. For example, the model 14 may be a neural network (NN), a support vector machine (SVM), a regression analysis model, a random forest, or another. Alternatively, the model 14 may be a k-nearest neighbor model that classifies the input data 18 with the k-nearest neighbor algorithm.

The measurement data 15 is obtained by a measurement device. The measurement data 15 may contain noise depending on the characteristics and usage environment of the measurement device. The measurement data 15 is collected for training the model 14. The data preprocessing apparatus 10 may receive the measurement data 15 directly from the measurement device connected thereto. Alternatively, the data preprocessing apparatus 10 may receive the measurement data 15 from a storage device or another information processing apparatus over a network. Yet alternatively, the data preprocessing apparatus 10 may read the measurement data 15 from a storage medium connected thereto.

The measurement data 15 may be time-series signal data representing time-series changes in amplitude, such as acceleration data obtained by an accelerometer, electrocardiogram data obtained by an electrocardiograph, or audio data obtained by a microphone. Alternatively, the measurement data 15 may be image data obtained by an image sensor. The measurement data 15 may correspond to a value of a specific response variable. For example, in the case where the model 14 is designed to perform binary classification into normal and abnormal, the measurement data 15 may represent a normal state. In addition, the measurement data 15 may be associated with a label that indicates a correct value of the response variable.

The measurement data 16 is data that is obtained by the measurement device and is of the same type as the measurement data 15. The measurement data 16, however, is collected after the training of the model 14. The measurement data 16 may be collected in the same way as the measurement data 15 or in a different way therefrom. The measurement data 16 may have a different tendency of noise from the measurement data 15. For example, the change in the tendency of noise may occur due to various factors such as the aging and replacement of the measurement device, a change in the location of the measurement device, and changes of electronic devices and architectures located in the vicinity of the measurement device. For example, a change in the frequencies of noise is considered as a change in the tendency of noise.

The label 16a indicates a correct value of the response variable corresponding to the measurement data 16. In the case where the model 14 is designed to perform binary classification into normal and abnormal, the label 16a indicates normal or abnormal. For example, the measurement data 16 obtained by the measurement device is confirmed by a human and then the label 16a is given by the human to the measurement data 16. The label 16a may be fed back each time the measurement data 16 is obtained. Alternatively, the label 16a may be fed back collectively at a later time after the measurement data 16 is accumulated.

The training data 17 is used for training the model 14. The training data 17 is generated by performing the preprocessing 13 on the measurement data 15. For example, the training data 17 is generated by a low-pass filter removing high-frequency noise from the measurement data 15. Note that the parameter 13a may be adjusted so that the preprocessing 13 substantially does not remove noise and the training data 17 and the measurement data 15 are thus identical. The parameter 13a used at the time of training the model 14 may be determined by trial and error by a human so as to generate the training data 17 appropriate for training the model 14 or may automatically be searched for through machine learning so as to improve the prediction accuracy of the model 14. For example, the parameter 13a is adjusted so that noise is sufficiently removed from the measurement data 15 and the substantial features of the measurement data 15 remain in the training data 17.

The input data 18 is generated by performing the preprocessing 13 on the measurement data 16. For example, the input data 18 is generated by a low-pass filter removing high-frequency noise from the measurement data 16. In principal, the same parameter 13a as used at the stage of training the model 14 is used in the preprocessing 13 of converting the measurement data 16 into the input data 18. Note that the use of the same parameter 13a as used at the training stage may fail to remove noise sufficiently from the measurement data 16 if the measurement data 16 has a different tendency of noise from the measurement data 15. To deal with this, the data preprocessing apparatus 10 may change the parameter 13a as described later.

The prediction result 19 is output from the model 14 having accepted an input of the input data 18. The input data 18 corresponds to the explanatory variable, and the prediction result 19 corresponds to the response variable. Since the label 16a indicating the correct value of the response variable is given, it is possible to evaluate the prediction accuracy of the model 14 by comparing the prediction result 19 with the label 16a. For example, accuracy is used as an evaluation value for the prediction accuracy. The accuracy is the ratio of the number of samples whose prediction results 19 agree with the label 16a to the total number of samples. The data preprocessing apparatus 10 takes measures to recover the prediction accuracy when the prediction accuracy decreases.

The processing unit 12 executes the training phase and the application phase. In the training phase, the processing unit 12 performs the preprocessing 13 based on the parameter 13a on the measurement data 15 to thereby generate the training data 17. The training data 17 is saved for possible later use in the application phase as described later. The processing unit 12 trains the model 14 using the training data 17. For example, in the case where the model 14 is a k-nearest neighbor model of performing binary classification, the model 14 calculates the distance between received input data and the training data 17, and determines that the input data is normal if the distance is less than or equal to a threshold and that the input data is abnormal if the distance exceeds the threshold.

In the application phase, the processing unit 12 performs the preprocessing 13 based on the same parameter 13a as used in the training phase on the measurement data 16 to thereby generate the input data 18. The processing unit 12 enters the input data 18 into the model 14 to generate the prediction result 19. For example, the prediction result 19 indicates whether the measurement data 16 is normal or abnormal. The processing unit 12 compares the prediction result 19 with the label 16a associated with the measurement data 16 and calculates the prediction accuracy of the model 14. For example, with respect to a plurality of samples being the measurement data 16, the processing unit 12 determines, for each sample, that its prediction result 19 is correct if the prediction result 19 agrees with the label 16a and that the prediction result 19 is incorrect if the prediction result 19 does not agree with the label 16a. The processing unit 12 calculates the ratio of samples having correct prediction results 19 among all samples as the prediction accuracy.

The processing unit 12 compares the calculated prediction accuracy with a threshold. The threshold is set to 90% or another in advance. In the case where the prediction accuracy is greater than or equal to the threshold, the processing unit 12 does not perform a recovery process for the prediction accuracy and keeps using the parameter 13a of the preprocessing 13. If the prediction accuracy is less than the threshold, however, the processing unit 12 performs the recovery process for the prediction accuracy. In the recovery process for the prediction accuracy, the processing unit 12 compares the training data 17 saved in the training phase with the input data generated from the measurement data 16 and changes the parameter 13a of the preprocessing 13 on the basis of the comparison result.

For example, while changing the parameter 13a and performing the preprocessing 13 on the measurement data 16, the processing unit 12 adjusts the parameter 13a so that the generated input data gets close to the training data 17. For example, the processing unit 12 calculates the distance between the generated input data and the training data 17 and adopts the parameter 13a that minimizes the distance. The processing unit 12 may search for the optimal parameter 13a with an optimization algorithm such as the gradient descent. Alternatively, the processing unit 12 may try some candidate values as the parameter 13a and adopt a candidate value that generates input data closest to the training data 17 from among the candidate values.

The change of the parameter 13a enables absorbing a change in the tendency of noise. For example, to change a cutoff frequency may result in removing noise of frequencies different from the training phase. At this time, the processing unit 12 does not need to retrain the model 14. The processing unit 12 then performs the preprocessing 13 using the new parameter 13a in the subsequent application phase. For example, the processing unit 12 performs the preprocessing 13 based on the new parameter 13a on new measurement data to generate input data, and enters the generated input data into the model 14 to generate a prediction result corresponding to the measurement data.

As described above, the data preprocessing apparatus 10 of the first embodiment performs the preprocessing 13 on the measurement data 15 to generate the training data 17 and trains the model 14 using the training data 17 in the training phase. The data preprocessing apparatus 10 then performs the preprocessing 13 on the measurement data 16 to generate the input data 18 and enters the input data 18 into the model 14 to generate the prediction result 19 in the application phase. When the prediction accuracy regarding the prediction result 19 decreases, the data preprocessing apparatus 10 changes the parameter 13a on the basis of a comparison between the saved training data 17 and the input data 18.

Even if the tendency of the measurement data 16 changes from the training phase due to changes in the characteristics and usage environment of the measurement device, the above-described approach is able to suppress influence of the change on the input data 18 to be entered into the model 14, which enables recovering the prediction accuracy of the model 14. In addition, the approach is able to keep using the model 14 without retraining it, which avoids an increase in cost such as the computational complexity and training time of the machine learning.

A machine learning apparatus of the second embodiment trains a model through machine learning and predicts a result corresponding to input data using the trained model. The machine learning apparatus of the second embodiment may be a client apparatus or a server apparatus. The machine learning apparatus may be called a computer or an information processing apparatus.

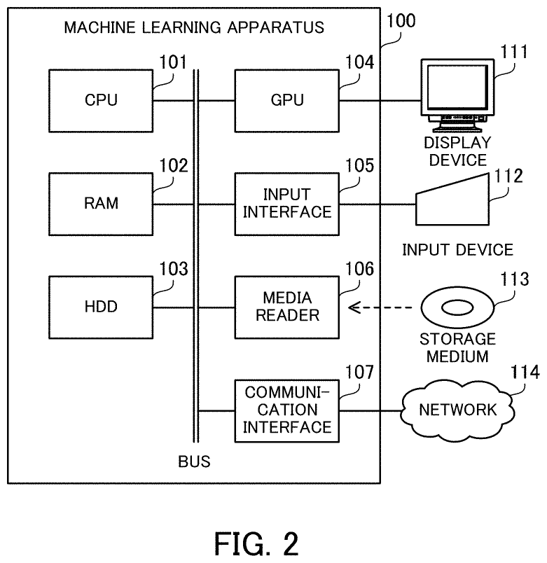

FIG. 2 illustrates an example of hardware configuration of a machine learning apparatus according to the second embodiment. The machine learning apparatus 100 includes a CPU 101, a RAM 102, an HDD 103, a GPU 104, an input interface 105, a media reader 106, and a communication interface 107. These units in the machine learning apparatus 100 are connected to a bus. The machine learning apparatus 100 corresponds to the data preprocessing apparatus 10 of the first embodiment. The CPU 101 corresponds to the processing unit 12 of the first embodiment. The RAM 102 or the HDD 103 corresponds to the storage unit 11 of the first embodiment.

The CPU 101 is a processor that executes program instructions. The CPU 101 loads at least part of a program and data from the HDD 103 to the RAM 102 and executes the program. The CPU 101 may include a plurality of processor cores, and the machine learning apparatus 100 may include a plurality of processors. A set of multiple processors may be called “a multiprocessor” or simply “a processor.”

The RAM 102 is a volatile semiconductor memory device that temporarily stores therein programs to be executed by the CPU 101 and data to be used by the CPU 101 in processing. The machine learning apparatus 100 may include a different type of memory device than RAM, or a plurality of memory devices.

The HDD 103 is a non-volatile storage device that stores therein software programs such as an operating system (OS), middleware, and application software, and data. The machine learning apparatus 100 may include a different type of storage device such as a flash memory or a solid state drive (SSD), or a plurality of storage devices.

The GPU 104 outputs images to a display device 111 connected to the machine learning apparatus 100 in accordance with instructions from the CPU 101. Examples of the display device 111 include a cathode ray tube (CRT) display, a liquid crystal display (LCD), an organic electro-luminescence (OEL) display, a projector, and any desired type of display device. Other than the display device 111, a printer or another output device may be connected to the machine learning apparatus 100.

The input interface 105 receives an input signal from an input device 112 connected to the machine learning apparatus 100. Examples of the input device 112 include a mouse, a touch panel, a touch pad, a keyboard, and any desired type of input device. A plurality of types of input devices may be connected to the machine learning apparatus 100.

The media reader 106 is a reading device that reads programs and data from a storage medium 113. Examples of the storage medium 113 include a magnetic disk, such as a flexible disk (FD) or an HDD, an optical disc, such as a compact disc (CD) or a digital versatile disc (DVD), a semiconductor memory device, and any desired type of storage medium. For example, the media reader 106 copies a program or data read from the storage medium 113 into the RAM 102, HDD 103, or another storage medium. The read program is executed by the CPU 101, for example. In this connection, the storage medium 113 may be a portable storage medium and may be used for distribution of programs and data. In addition, the storage medium 113 and HDD 103 may be called computer-readable storage media.

The communication interface 107 is connected to a network 114 and communicates with other apparatuses over the network 114. The communication interface 107 may be a wired communication interface that is connected to a switch, a router, or another wired communication device or may be a wireless communication interface that is connected to a base station, an access point, or another wireless communication device.

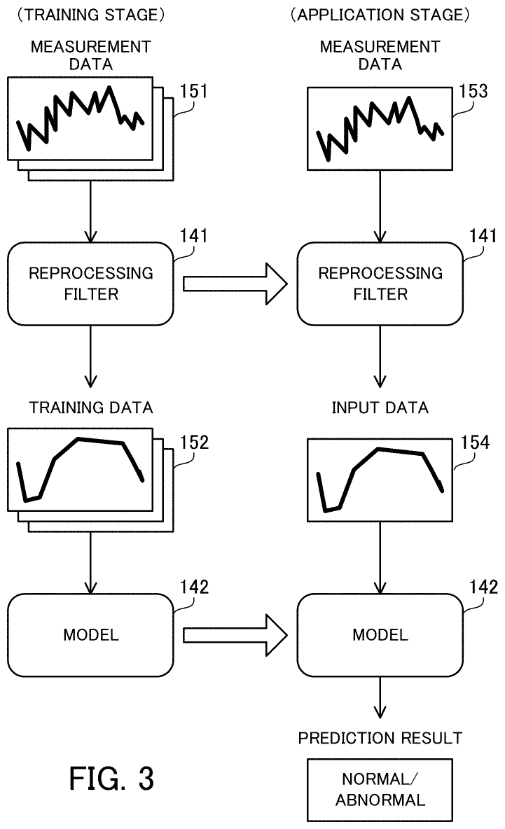

The following describes a sequence of training and applying a model. FIG. 3 illustrates an example of a sequence of training and applying a model. The machine learning apparatus 100 collects measurement data 151. The measurement data 151 is past data obtained by a measurement device. The measurement data 151 contains noise caused by the characteristics and usage environment of the measurement device. The noise may occur due to the structure of the measurement device itself or due to the electromagnetic waves of electronic devices located in the surroundings. As the measurement data 151, a plurality of samples obtained from different targets are collected. As described later, it is assumed that the second embodiment mainly uses electrocardiogram data obtained by an electrocardiograph as the measurement data 151. A plurality of electrocardiogram samples obtained from different patients in the past are collected. The electrocardiogram samples collected as the measurement data 151 are normal samples determined by a human as representing normal electrocardiograms.

The machine learning apparatus 100 enters the measurement data 151 into a preprocessing filter 141 to generate training data 152. The preprocessing filter 141 is designed to remove noise from the measurement data 151. As described later, it is assumed that the second embodiment mainly uses a low-pass filter of removing high-frequency noise as the preprocessing filter 141. The behavior of the low-pass filter depends on a cutoff frequency indicating an upper limit of frequencies that the low-pass filter passes. The cutoff frequency is adjusted by trial and error by an operator at the training stage. It is assumed that a plurality of electrocardiogram samples from which high-frequency noise has been removed are mainly used as the training data 152.

The machine learning apparatus 100 trains a model 142 using the training data 152. The model 142 is a classifier that classifies input data into a plurality of classes. The model 142 may be a neural network, a support vector machine, a regression analysis model, a random forest, or another. As described later, it is assumed that the second embodiment mainly uses, as the model 142, a k-nearest neighbor model of classifying input data into normal and abnormal with the k-nearest neighbor algorithm. This k-nearest neighbor model calculates the distance between an entered electrocardiogram sample and a normal sample that is the training data 152, and determines that the electrocardiogram sample is normal if the distance is less than or equal to a threshold and that the electrocardiogram sample is abnormal if the distance exceeds the threshold. Such a model 142 is usable at a medical site. Whether an electrocardiogram is normal or abnormal is determined for diagnosing a patient's disease.

After training the model 142, the machine learning apparatus 100 obtains measurement data 153. The measurement data 153 is measured by the measurement device after the training of the model 142. The measurement data 153 contains noise depending on the characteristics and usage environment of the measurement device. In addition, the machine learning apparatus 100 obtains a label that is fed back with respect to the measurement data 153 after the measurement data 153 is obtained. The label indicates a correct class to which the measurement data 153 belongs. As with the measurement data 151, it is assumed that electrocardiogram data obtained by an electrocardiograph is mainly used as the measurement data 153. The label indicates a result of determining by a human whether the electrocardiogram is normal or abnormal.

The machine learning apparatus 100 enters the measurement data 153 into the preprocessing filter 141 to generate input data 154. The preprocessing filter 141 is designed to remove noise from the measurement data 153. The preprocessing filter 141 used here is the same as used at the training stage and is, for example, a low-pass filter with the same cutoff frequency as used at the training stage. It is assumed that, as the input data 154, an electrocardiogram sample from which high-frequency noise has been removed is mainly used. The machine learning apparatus 100 enters the input data 154 into the model 142 and outputs a prediction result regarding a class to which the input data 154 belongs. For example, the model 142 calculates the distance between the electrocardiogram sample being the input data 154 and a normal sample being the training data 152, and determines that the electrocardiogram sample is normal if the distance is less than or equal to a threshold and that the electrocardiogram sample is abnormal if the distance exceeds the threshold. It is possible to evaluate the prediction accuracy of the model 142 by comparing the prediction result with the label.

In the second embodiment, it is assumed that the distribution of characteristics of ideal measurement data without noise does not change between the training and application stages, i.e., concept drift does not occur. It is otherwise assumed that, even if concept drift occurs, the change is sufficiently gentle and the tendency of the change is known. For example, it is assumed that the relationship between an electrocardiogram waveform without noise and a classification as normal or abnormal does not change between the training and application stages.

It is noted that the distribution of noise contained in measurement data may change between the training and application stages due to the replacement and aging of a measurement device, a change in the location of the measurement device, and changes of electronic devices located in the vicinity of the measurement device. If this change happens, the input data after the preprocessing may have changed characteristics accordingly, and the prediction accuracy of the model may thus decrease.

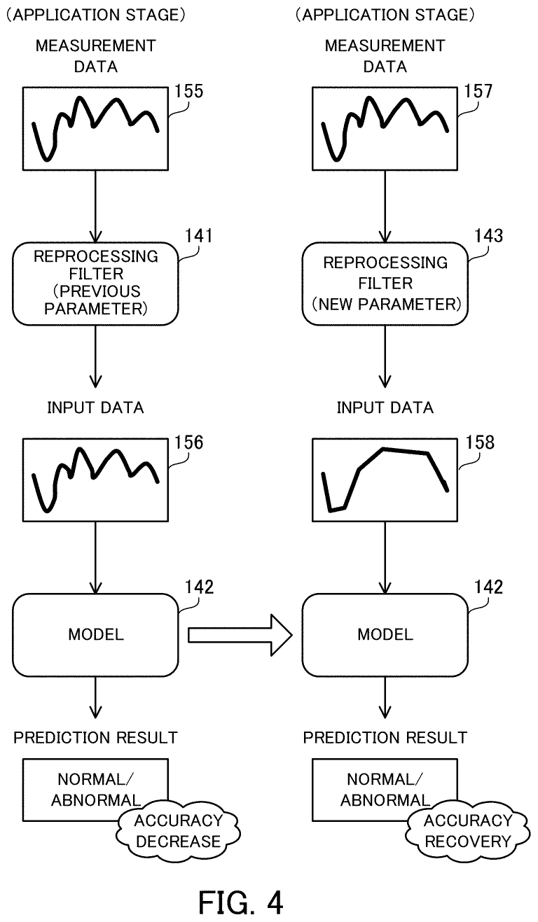

FIG. 4 illustrates an example of a sequence of decrease due to noise and recovery in prediction accuracy. After training the model 142, the machine learning apparatus 100 obtains measurement data 155. The measurement data 155 contains noise caused by the characteristics and usage environment of the measurement device. The measurement data 155 has a different tendency of noise from the measurement data 151 used at the training stage. For example, the frequencies of noise contained in electrocardiogram data have changed.

This situation has a possibility that the use of the same preprocessing filter 141 as used at the training stage may fail to remove the noise from the measurement data 155 properly. Therefore, input data 156 generated from the measurement data 155 by the preprocessing filter 141 may fail to match the distribution of the training data 152 used for training the model 142. For example, because the cutoff frequency becomes inappropriate, large noise may remain in the input data 156 or the input data 156 may have an excessively smoothed signal waveform.

As a result, the prediction accuracy regarding the prediction result output from the model 142 having accepted the input data 156 may be lower than the prediction accuracy obtained at the time of training the model 142. For example, large noise remaining in the input data 156 increases a risk of erroneously determining that the normal electrocardiogram data is abnormal. One of methods to recover the prediction accuracy is to collect newer measurement data than the measurement data 151 and to train a new model, which replaces the model 142, using the new measurement data. The retraining of the model, however, costs a lot in terms of computational complexity and training time.

Here, instead of retraining the model, the machine learning apparatus 100 changes the preprocessing filter to deal with the change in the tendency of noise. More specifically, the machine learning apparatus 100 saves the training data 152 after the preprocessing, used for training the model 142. The machine learning apparatus 100 changes a parameter of the preprocessing filter such that the input data obtained by converting the measurement data 155 gets closer to the saved training data 152. For example, the machine learning apparatus 100 calculates the distance between the input data having passed through the preprocessing filter and the training data 152 and optimizes the parameter of the preprocessing filter so as to minimize the distance.

By doing so, the preprocessing filter 141 is changed to a preprocessing filter 143 having a different parameter from the preprocessing filter 141. For example, the cutoff frequency of the low-pass filter is changed. After that, the machine learning apparatus 100 obtains measurement data 157. The measurement data 157 contains the same tendency of noise as the measurement data 155. The machine learning apparatus 100 enters the measurement data 157 into the preprocessing filter 143 to convert the measurement data 157 into input data 158. It is expected that the input data 158 is obtained by removing noise from the measurement data 157. The characteristics of the input data 158 match those of the training data 152.

The machine learning apparatus 100 enters the input data 158 into the model 142 to obtain a prediction result. It is expected that the prediction accuracy of the model 142 is recovered to the same level as the prediction accuracy of the model 142 at the time of training. This is because the characteristics of the input data 158 entered into the model 142 are sufficiently close to those of the training data 152 used for training the model 142.

In this connection, in the case where the tendency of noise has a large change, there is a possibility that the input data obtained by converting the measurement data 155 does not get closer to the training data 152 even if the parameter of the preprocessing filter is set to any value. In this case, the machine learning apparatus 100 may output a warning indicating a suggestion to retrain the model. For example, the machine learning apparatus 100 may be designed to calculate the distance between input data having passed through the optimized preprocessing filter 143 and the training data 152 and to output a warning if the calculated distance exceeds a predetermined threshold.

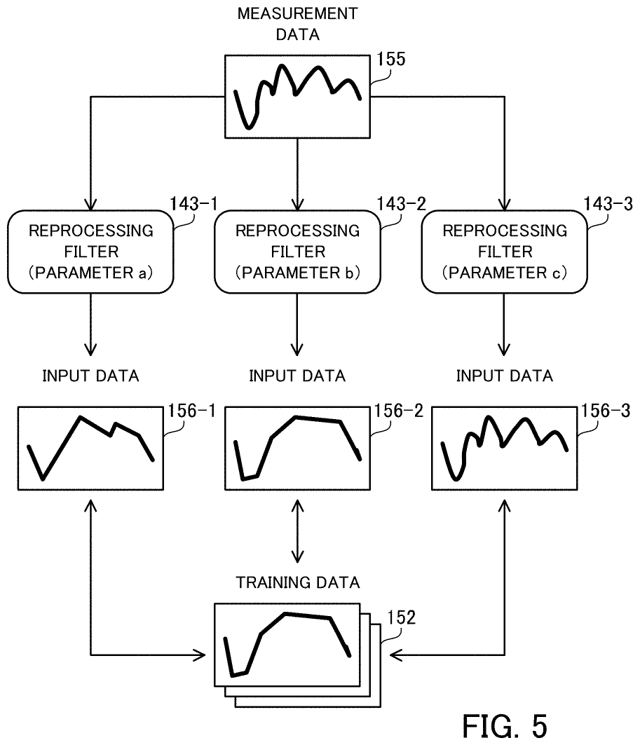

FIG. 5 illustrates an example of searching for a parameter of a preprocessing filter. To search for a parameter of a preprocessing filter, the machine learning apparatus 100 may use an optimization algorithm such as the gradient descent. Alternatively, the machine learning apparatus 100 may try some parameters and adopt a parameter that yields a minimum distance from among the parameters. In the following, the latter method will be described.

The machine learning apparatus 100 creates preprocessing filters 143-1, 143-2, and 143-3 having different parameters. The preprocessing filter 143-1 has a parameter a, the preprocessing filter 143-2 has a parameter b, and the preprocessing filter 143-3 has a parameter c. For example, the preprocessing filters 143-1, 143-2, and 143-3 are low-pass filters having different cutoff frequencies. For example, the preprocessing filter 143-1 is a strong filter with a low cutoff frequency, the preprocessing filter 143-2 is a moderate filter with a medium cutoff frequency, and the preprocessing filter 143-3 is a weak filter with a high cutoff frequency. The machine learning apparatus 100 may be designed to select three of predetermined cutoff frequencies such as 25 Hz, 35 Hz, 75 Hz, 100 Hz, and 150 Hz.

The machine learning apparatus 100 enters the measurement data 155 into the preprocessing filter 143-1 to generate input data 156-1. The machine learning apparatus 100 enters the measurement data 155 into the preprocessing filter 143-2 to generate input data 156-2. The machine learning apparatus 100 also enters the measurement data 155 into the preprocessing filter 143-3 to generate input data 156-3. Then, the machine learning apparatus 100 calculates the distances between each input data 156-1, 156-2, and 156-3 and the training data 152. In the case where the training data 152 includes a plurality of samples, the distance between the input data 156-1 and the training data 152 may be defined by the distance between the input data 156-1 and a sample closest to the input data 156-1 among the plurality of samples. Similarly, the distance between the input data 156-2 and the training data 152 may be defined by the distance between the input data 156-2 and a sample closest to the input data 156-2.

The machine learning apparatus 100 detects input data that has the minimum distance to the training data 152 from among the input data 156-1, 156-2, and 156-3. It is now assumed that the input data 156-2 has the minimum distance. Then, the machine learning apparatus 100 adopts the preprocessing filter 143-2 used for generating the input data 156-2. That is, the machine learning apparatus 100 changes the parameter of the preprocessing filter to the parameter b. For the subsequently obtained measurement data, the preprocessing filter 143-2 having the parameter b is used.

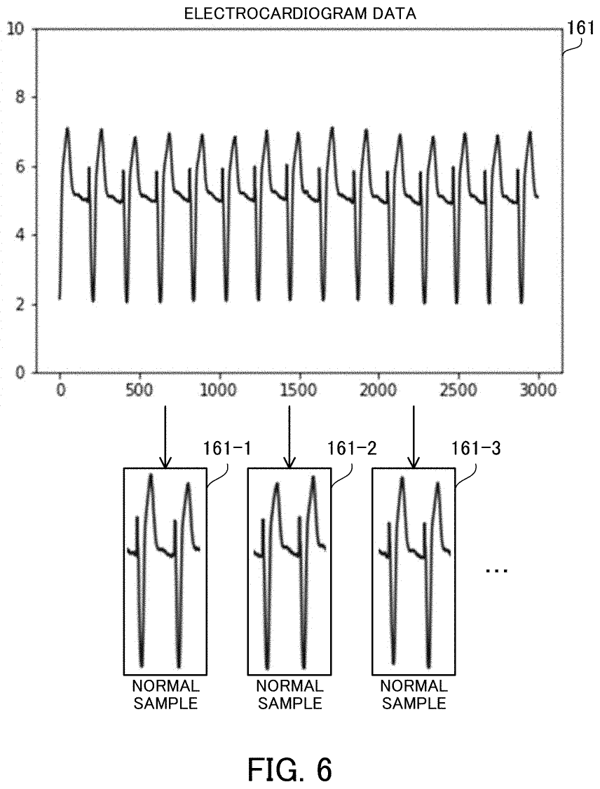

The following describes an example of using electrocardiogram data as measurement data. FIG. 6 illustrates an example of generating training data. The machine learning apparatus 100 obtains electrocardiogram data 161 obtained in the past, in order to train a model. The electrocardiogram data 161 represents a normal electrocardiogram. The electrocardiogram data 161 has a waveform with a repeated predetermined pattern representing heart beats. The machine learning apparatus 100 extracts waveforms for a predetermined number of periods, such as two periods, from the electrocardiogram data 161, and generates normal samples 161-1, 161-2, 161-3, representing the extracted waveforms. The plurality of normal samples are used as training data for training a model. The training data preferably includes normal samples obtained from different patients.

In generating the normal samples 161-1, 161-2, 161-3, . . . from the electrocardiogram data 161, the time duration and amplitude are normalized. For example, the machine learning apparatus 100 stretches or compresses the waveforms of predetermined periods extracted from the electrocardiogram data 161 in the time domain so that the normal samples 161-1, 161-2, 161-3, . . . have the same time duration. In addition, for example, the machine learning apparatus 100 stretches or compresses the extracted waveforms of predetermined periods in the amplitude domain so that the normal samples 161-1, 161-2, 161-3, . . . have the same fluctuation width of signal level. The normalization of the time duration and amplitude are performed in the preprocessing. In this connection, in the case of training a model that calculates the distance between a normal sample and an input sample while automatically adjusting differences in time duration and amplitude, the time duration and amplitude do not need to be normalized at the time of generating training data.

In addition, in generating the normal samples 161-1, 161-2, 161-3, . . . from the electrocardiogram data 161, a low-pass filter is used to remove high-frequency noise. The removal of high-frequency noise is performed in the preprocessing. The cutoff frequency of the low-pass filter is determined by trial and error by an operator of the model training. In the following, assume that, for simple description, the noise of the electrocardiogram data 161 is sufficiently small and training data is generated without removing high-frequency noise using the low-pass filter. The omission of removal of high-frequency noise is equivalent to the setting of a sufficiently high cutoff frequency.

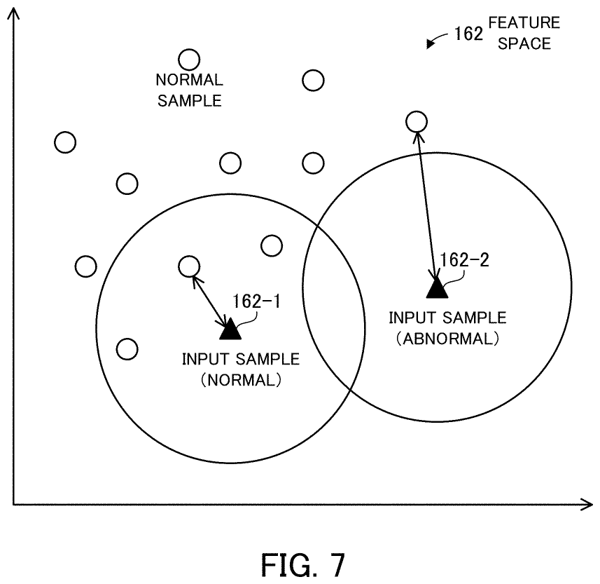

FIG. 7 illustrates an example of abnormality detection by a k-nearest neighbor model. The machine learning apparatus 100 creates a k-nearest neighbor model of classifying input samples into normal and abnormal with the k-nearest neighbor algorithm, using the normal samples 161-1, 161-2, 161-3, . . . that are training data. In the second embodiment, only a normal sample closest to an input sample influences a determination result. Therefore, the k-nearest neighbor model of the second embodiment may be called a nearest neighbor model of classifying an input sample with the nearest neighbor algorithm.

More specifically, the machine learning apparatus 100 creates a feature space 162 in which the normal samples 161-1, 161-2, 161-3, . . . being training data are placed. When receiving an input sample, the k-nearest neighbor model searches for a normal sample whose distance to the input sample is less than or equal to a predetermined threshold (for example, 0.3) in the feature space 162. In the case where at least one normal sample exists within the predetermined distance from the input sample, the k-nearest neighbor model determines that the input sample is normal. If no normal sample exists within the predetermined distance from the input sample, the k-nearest neighbor model determines that the input sample is abnormal.

For example, the input sample 162-1 in FIG. 7 is determined to be normal because at least one normal sample exists within the predetermined distance. The input sample 162-2 in FIG. 7 is, however, determined to be abnormal because no normal sample exists within the predetermined distance. For example, the k-nearest neighbor model calculates the distance between an input sample and each of the plurality of normal samples and determines whether the calculated distances are less than or equal to the threshold. The k-nearest neighbor model determines that the input sample is normal if the minimum distance is less than or equal to the threshold and that the input sample is abnormal if the minimum distance exceeds the threshold. Note that the machine learning apparatus 100 may be designed to generate an index for estimating the distance to an input sample so as to efficiently find normal samples whose distances to the input sample are possibly less than or equal to the threshold. With this, the k-nearest neighbor model does not need to calculate the distance to each normal sample.

Each of the input samples and normal samples is time-series data representing a signal waveform. The distance between one input sample and one normal sample represents the degree of similarity between their signal waveforms. A smaller distance means a higher similarity between two signal waveforms, whereas a greater distance means a greater difference between two signal waveforms. For example, the k-nearest neighbor model calculates the absolute value of the difference in signal level between two signal waveforms at each time point along the time axis, and defines the mean value as the distance. Alternatively, for example, the k-nearest neighbor model calculates the square of the difference in signal level between two signal waveforms at each time point along the time axis, and defines the square root of the mean value as the distance. In addition, the k-nearest neighbor model may be designed to calculate the distance between two signal waveforms while modifying a shift in the time domain between these signal waveforms using dynamic programming such as dynamic time warping (DTW).

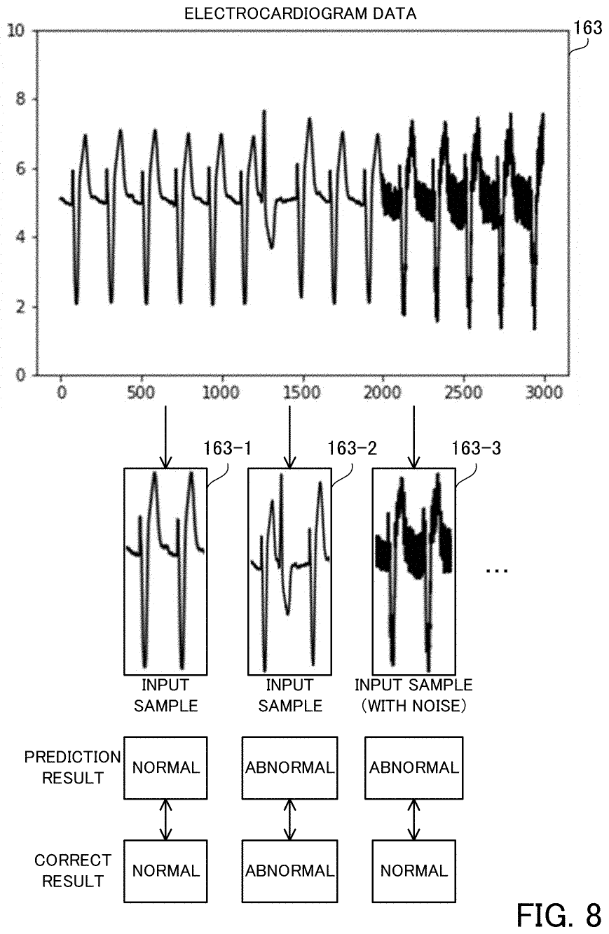

FIG. 8 illustrates an example of erroneous detection with respect to an input sample with noise. After training the k-nearest neighbor model, the machine learning apparatus 100 obtains electrocardiogram data 163 captured after the training of the k-nearest neighbor model. The electrocardiogram data 163 represents an electrocardiogram that may be partly normal and partly abnormal. In addition, the electrocardiogram data 163 may contain noise of different frequencies from the electrocardiogram data 161 used for training the k-nearest neighbor model. Such a change in the tendency of noise may occur due to the replacement and aging of an electrocardiograph, a change in the location of the electrocardiograph, a change in the surrounding environment of the electrocardiograph, and others.

The machine learning apparatus 100 extracts waveforms for a predetermined number of periods, such as two periods, from the electrocardiogram data 163, and performs the same preprocessing as in the training stage on the extracted waveforms to thereby generate input samples 163-1, 163-2, 163-3, . . . . The time duration and amplitude of each input sample 161-1, 161-2, 161-3, are normalized. For example, the machine learning apparatus 100 stretches or compresses the extracted waveforms of predetermined periods in the time domain so that the input samples 161-1, 161-2, 161-3, . . . have the same time duration as the normal samples 161-1, 161-2, 161-3, In addition, for example, the machine learning apparatus 100 stretches or compresses the extracted waveforms of predetermined periods in the amplitude domain so that the input samples 161-1, 161-2, 161-3, . . . have the same fluctuation width of signal level as the normal samples 161-1, 161-2, 161-3, In this connection, whether to normalize the time durations and amplitudes of the input samples 163-1, 163-2, 163-3, . . . depends on a model used.

In addition, high-frequency noise is removed from the input samples 163-1, 163-2, 163-3, . . . using a low-pass filter. The low-pass filter is set to have the same cutoff frequency as used at the model training stage. In this connection, as described above, it is assumed that, for simple description, the removal of high-frequency noise is not performed by the low-pass filter at the model training stage and the removal of high-frequency noise using the low-pass filter is not performed here as well. The omission of removal of the high-frequency noise is equivalent to the setting of a sufficiently high cutoff frequency.

The machine learning apparatus 100 enters the generated input samples 163-1, 163-2, 163-3, . . . to the k-nearest neighbor model to determine whether each input sample is normal or abnormal. The machine learning apparatus 100 determines that the input sample 163-1 is normal, the input sample 163-2 is abnormal, and the input sample 163-3 is abnormal. The machine learning apparatus 100 outputs these prediction results with respect to the input samples 163-1, 163-2, 163-3. For example, the machine learning apparatus 100 displays the prediction results on the display device 111.

The correct results are that the input sample 163-1 is normal, the input sample 163-2 is abnormal, and the input sample 163-3 is normal. The input sample 163-1 does not contain noise that is not expected at the model training stage, and therefore the k-nearest neighbor model correctly determines that the normal electrocardiogram waveform is normal. Similarly, the input sample 163-2 does not contain noise that is not expected at the model training stage, and therefore the k-nearest neighbor model correctly determines that the abnormal electrocardiogram waveform is abnormal. However, the input sample 163-3 contains high-frequency noise that is not expected at the model training stage, and therefore the k-nearest neighbor model erroneously determines that the normal electrocardiogram waveform is abnormal.

This erroneous determination regarding the input sample 163-3 decreases the accuracy of the k-nearest neighbor model, i.e., decreases the prediction accuracy. The accuracy is the ratio of the number of input samples with correct prediction results of normal or abnormal to the total number of input samples entered into the k-nearest neighbor model. The latest prediction accuracy is evaluated by calculating the accuracy with respect to a predetermined number of recent input samples. When the prediction accuracy of the k-nearest neighbor model falls below a threshold (for example, 90%), the machine learning apparatus 100 makes an attempt to recover the prediction accuracy by changing a parameter of the low-pass filter.

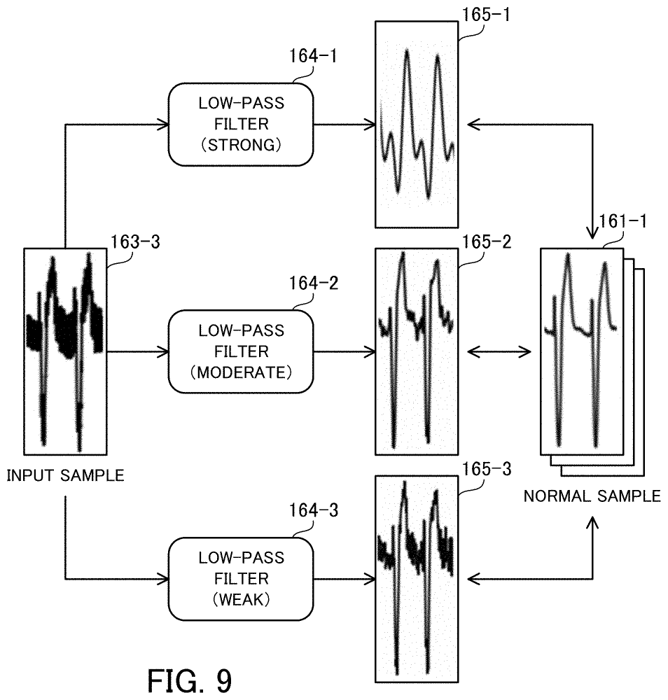

FIG. 9 illustrates an example of searching for a parameter of a low-pass filter. The machine learning apparatus 100 selects one or more input samples that have caused a decrease in prediction accuracy from input samples entered into the k-nearest neighbor model. As is the above-described input sample 163-3, the input samples that have caused the decrease in the prediction accuracy are ones that are determined to be abnormal by the k-nearest neighbor model from among input samples given a label indicating normal. That is, these input samples have high probability that the input samples are correctly determined to be normal if high-frequency noise is properly removed therefrom by a low-pass filter.

It may be said that an input sample that has caused the decrease in the prediction accuracy is detected based on a comparison between the input sample having passed through the low-pass filter and a normal sample that is training data having passed through the low-pass filter. In the case where the distance between a normal sample having passed through the low-pass filter and an input sample having passed through the low-pass filter and being normal exceeds a threshold, the input sample is determined to have caused the decrease in the prediction accuracy.

In the case where a predetermined number of recent input samples include two or more input samples erroneously determined to be abnormal, the machine learning apparatus 100 may select one of these input samples. The machine learning apparatus 100 may select one input sample randomly or under predetermined criteria. For example, the machine learning apparatus 100 may select an input sample whose distance to the training data, calculated by the k-nearest neighbor model, i.e., whose minimum distance to a normal sample most similar thereto is the greatest. Such an input sample is said to contain the greatest noise. Alternatively, the machine learning apparatus 100 may select all of the two or more input samples erroneously determined.

In addition, the machine learning apparatus 100 creates a plurality of low-pass filters having different cutoff frequencies. For example, the machine learning apparatus 100 creates some low-pass filters such as low-pass filters 164-1, 164-2, and 164-3. The low-pass filter 164-1 is a strong filter that has a low cutoff frequency and allows a few frequency components to pass through. The low-pass filter 164-2 is a moderate filter that has a medium cutoff frequency and allows medium frequency components to pass through. The low-pass filter 164-3 is a weak filter that has a high cutoff frequency and allows many frequency components to pass through. A cutoff frequency is set to 25 Hz, 35 Hz, 75 Hz, 100 Hz, 150 Hz, or another.

A low-pass filter for time-series signal data may be implemented by using an FIR filter or IIR filter. The FIR filter holds a predetermined number of recent input signals, and outputs, as the latest output signal, a signal obtained by multiplying each of the latest input signal and a predetermined number of past input signals by a filter coefficient and summing the multiplication results. The number of input signals held therein, that is, a storage time may be specified as a filter order. By adjusting the filter order and filter coefficient, low-pass filters with different frequency characteristics are created. The IIR filter holds a predetermined number of past output signals in addition to a predetermined number of past input signals. The IIR filter outputs, as the latest output signal, a signal obtained by multiplying each of the latest input signal, a predetermined number of past input signals, and a predetermined number of past output signals by a filter coefficient and summing the multiplication results.

The machine learning apparatus 100 is able to create an FIR filter or IIR filter that operates as a low-pass filter, using a mathematics library. For example, when accepting specification of a filter order and cutoff frequency, the mathematics library may automatically create an FIR filter or IIR filter having an appropriate filter coefficient. In addition to the filter order and cutoff frequency, amplitudes at frequencies around the cutoff frequency may be specified as information indicating amplitude attenuation characteristics.

The machine learning apparatus 100 enters an unfiltered sample, which has not passed through a low-pass filter, corresponding to the selected input sample into each of the low-pass filters 164-1, 164-2, and 164-3. Please note that input samples having not passed through a low-pass filter have been entered into the k-nearest neighbor model and have caused the decrease in the prediction accuracy, and therefore the input sample 163-3 is entered into the low-pass filters 164-1, 164-2, and 164-3 as it is. The machine learning apparatus 100 enters the input sample 163-3 into the low-pass filter 164-1 to generate a sample 165-1. The machine learning apparatus 100 enters the input sample 163-3 into the low-pass filter 164-2 to generate a sample 165-2. The machine learning apparatus 100 enters the input sample 163-3 into the low-pass filter 164-3 to generate a sample 165-3.

The machine learning apparatus 100 then calculates the distances between each generated sample 165-1, 165-2, and 165-3 and the training data including the normal samples 161-1, 161-2, 161-3, The calculated distances correspond to distances calculated by the k-nearest neighbor model taking each sample 165-1, 165-2, 165-3, . . . as an input sample entered into the k-nearest neighbor model. Here, the distance calculated with respect to a sample is the minimum distance calculated between the sample and a normal sample that is the most similar to the sample among the normal samples 161-1, 161-2, 161-3, . . . .

The machine learning apparatus 100 determines a sample having the minimum distance to the training data from among the samples 165-1, 165-2, and 165-3. The machine learning apparatus 100 then adopts a low-pass filter used for generating the found sample as a low-pass filter for use for subsequently obtained electrocardiogram data. Assume now that the sample 165-2 among the samples 165-1, 165-2, and 165-3 has the minimum distance to the training data. The machine learning apparatus 100 then selects the low-pass filter 164-2 among the low-pass filters 164-1, 164-2, and 164-3. This means selecting the parameters of the low-pass filter 164-2 such as a cutoff frequency and a filter order.

In this connection, in the case of selecting two or more input samples that have caused the decrease in the prediction accuracy, the machine learning apparatus 100 may select a low-pass filter that minimizes the average distance of two or more distances calculated with respect to these two or more input samples. Alternatively, the machine learning apparatus 100 may select a low-pass filter that minimizes the worst value (maximum distance) of the two or more distances calculated with respect to the two or more input samples. Alternatively, the machine learning apparatus 100 may use an optimization algorithm such as the gradient descent to search for parameters that yield the minimum distance by repeatedly calculating the distance between a filtered sample and training data while changing the parameters of the low-pass filter.

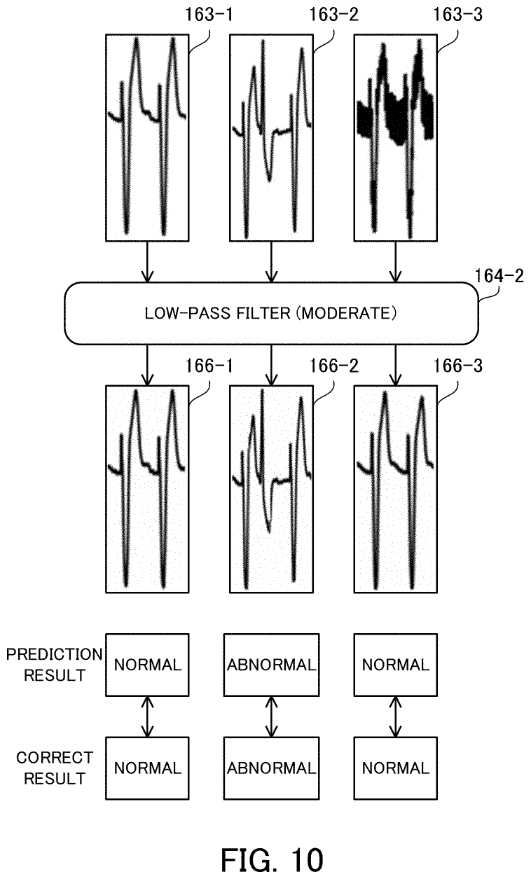

FIG. 10 illustrates an example of applying a first low-pass filter. The machine learning apparatus 100 adopts the low-pass filter 164-2 as described above. The following describes an improvement of the prediction accuracy for the electrocardiogram data 163 with the use of the low-pass filter 164-2, without the need of retraining the k-nearest neighbor model.

The machine learning apparatus 100 enters the input sample 163-1 that has not passed through a low-pass filter into the low-pass filter 164-2 to convert it to an input sample 166-1. The machine learning apparatus 100 enters the input sample 163-2 that has not passed through a low-pass filter into the low-pass filter 164-2 to convert it to an input sample 166-2. The machine learning apparatus 100 enters the input sample 163-3 that has not passed through a low-pass filter into the low-pass filter 164-2 to convert it to input sample 166-3. The machine learning apparatus 100 enters the input samples 166-1, 166-2, and 166-3 into the k-nearest neighbor model to determine whether each of the input samples 166-1, 166-2, and 166-3 is normal or abnormal.

The input sample 163-1 does not contain high-frequency noise, and the input sample 166-1 does not contain high-frequency noise. The input sample 166-1 represents a normal electrocardiogram waveform, and its characteristics match those of the training data. Therefore, the machine learning apparatus 100 correctly determines that the normal input sample 166-1 is normal. In addition, the input sample 163-2 does not contain high-frequency noise, and the input sample 166-2 does not contain high-frequency noise. The input sample 166-2 represents an abnormal electrocardiogram waveform. Therefore, the machine learning apparatus 100 correctly determines that the abnormal input sample 166-2 is abnormal.

The input sample 163-3 contains high-frequency noise, but the input sample 166-3 does not contain high-frequency noise because the high-frequency noise is properly removed by the low-pass filter 164-2. The input sample 166-3 represents a normal electrocardiogram waveform and its characteristics match those of the training data. Therefore, the machine learning apparatus 100 correctly determines that the normal input sample 166-3 is normal. As described above, it is possible to recover the prediction accuracy of the k-nearest neighbor model by adjusting the parameters so that an input sample having passed through the low-pass filter gets closer to the training data used for training the k-nearest neighbor model.

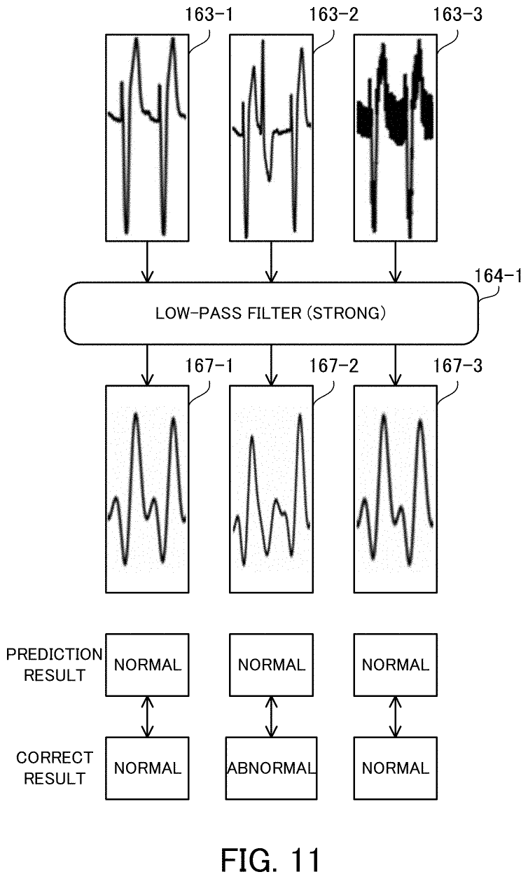

FIG. 11 illustrates an example of applying a second low-pass filter.

Consider the case of adopting the low-pass filter 164-1. The low-pass filter 164-1 have a too low cutoff frequency, and therefore an input sample having passed through the low-pass filter 164-1 has greatly different characteristics from the training data. This means that the prediction accuracy of the k-nearest neighbor model is not sufficiently recovered.

The machine learning apparatus 100 enters the input sample 163-1 that has not passed through a low-pass filter into the low-pass filter 164-1 to convert it to an input sample 167-1. The machine learning apparatus 100 enters the input sample 163-2 that has not passed through a low-pass filter into the low-pass filter 164-1 to convert it to an input sample 167-2. The machine learning apparatus 100 enters the input sample 163-3 that has not passed through a low-pass filter into the low-pass filter 164-1 to convert it to an input sample 167-3. The machine learning apparatus 100 enters the input samples 167-1, 167-2, and 167-3 into the k-nearest neighbor model to determine whether each of the input samples 167-1, 167-2, and 167-3 is normal or abnormal.

The input sample 167-1 does not contain high-frequency noise, and the machine learning apparatus 100 correctly determines that the normal input sample 167-1 is normal. The input sample 167-3 is obtained by removing high-frequency noise, and therefore the machine learning apparatus 100 correctly determines that the normal input sample 167-3 is normal. Although the input sample 167-2 does not contain high-frequency noise, the input sample 167-2 has lost abnormal characteristics of electrocardiogram waveform due to the excessive filtering. Therefore, the machine learning apparatus 100 erroneously determines that the abnormal input sample 167-2 is normal. As described above, the prediction accuracy is not recovered sufficiently depending on how to adjust the parameters of the low-pass filter.

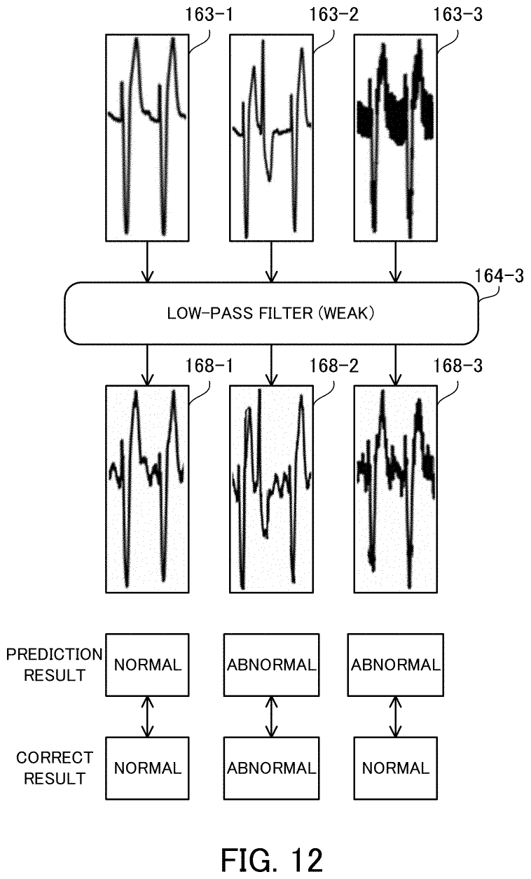

FIG. 12 illustrates an example of applying a third low-pass filter. Consider now the case of adopting the low-pass filter 164-3. Since the low-pass filter 164-3 has a too high cutoff frequency, input samples having passed through the low-pass filter 164-3 still contain high-frequency noise.

The machine learning apparatus 100 enters the input sample 163-1 that has not passed through a low-pass filter into the low-pass filter 164-3 to convert it to an input sample 168-1. The machine learning apparatus 100 enters the input sample 163-2 that has not passed through a low-pass filter into the low-pass filter 164-3 to convert it to an input sample 168-2. The machine learning apparatus 100 enters the input sample 163-3 that has not passed through a low-pass filter into the low-pass filter 164-3 to convert it to an input sample 168-3. The machine learning apparatus 100 enters the input samples 168-1, 168-2, and 168-3 into the k-nearest neighbor model to determine whether each of the input samples 168-1, 168-2, and 168-3 is normal or abnormal.

The input sample 168-1 does not contain high-frequency noise, and the machine learning apparatus 100 correctly determines that the normal input sample 168-1 is normal. The input sample 168-2 does not contain high-frequency noise but still has abnormal characteristics of electrocardiogram waveform, and therefore the machine learning apparatus 100 correctly determines that the abnormal input sample 168-2 is abnormal. The input sample 168-3 still contains high-frequency noise, and therefore the machine learning apparatus 100 erroneously determines that the normal input sample 168-3 is abnormal. As described above, the prediction accuracy is not recovered sufficiently depending on how to adjust the parameters of the low-pass filter.

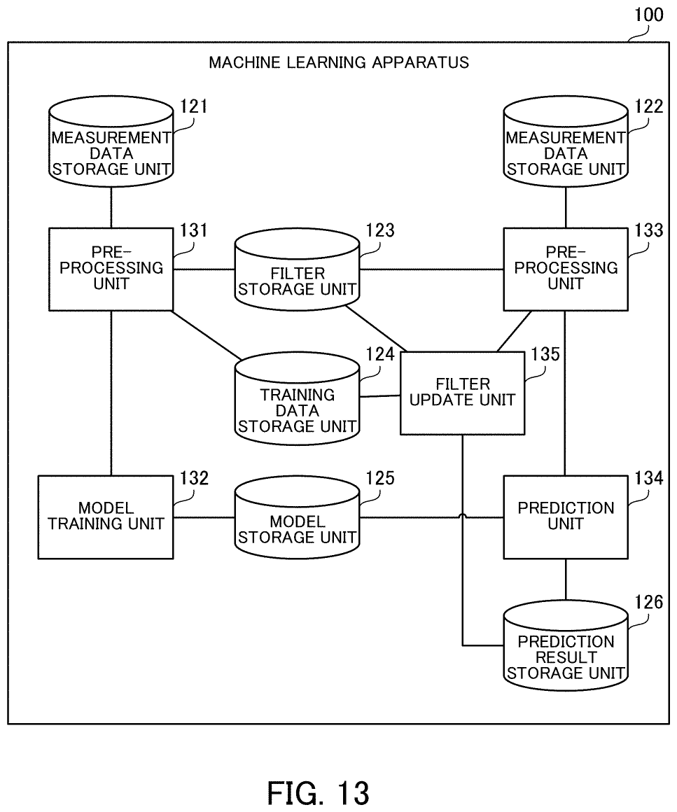

The following describes the functions of the machine learning apparatus 100. FIG. 13 is a block diagram illustrating an example of functions of the machine learning apparatus. The machine learning apparatus 100 has measurement data storage units 121 and 122, a filter storage unit 123, a training data storage unit 124, a model storage unit 125, and a prediction result storage unit 126. These storage units are implemented by using a storage space of the RAM 102 or HDD 103, for example. The machine learning apparatus 100 also has preprocessing units 131 and 133, a model learning unit 132, a prediction unit 134, and a filter update unit 135. These processing units are implemented by the CPU 101 executing a program, for example.

The measurement data storage unit 121 holds measurement data that is used for training a model. The measurement data is obtained by a measurement device and may contain noise depending on the hardware characteristics and usage environment of the measurement device. The measurement data may be time-series data or spatial data at a certain time point. For example, the measurement data is image data obtained by an imaging device, audio data obtained by a microphone, walking data obtained by an accelerometer, electrocardiogram data obtained by an electrocardiograph, or another. The measurement data may be given a label indicating a correct classification class. In this connection, in the case where only measurement data belonging to a specific class is used as training data, there is no need to use the label.

The measurement data storage unit 122 holds measurement data obtained after the measurement data stored in the measurement data storage unit 121. The measurement data in the measurement data storage unit 122 is of the same type as that in the measurement data storage unit 121 and is obtained after the application of the model starts. Note that the measurement data in the measurement data storage unit 122 may contain noise having a different tendency from the noise of the measurement data used for the training due to changes in the hardware characteristics and usage environment of the measurement device. The measurement data is given a label indicating a correct classification class. This label is fed back for the measurement data at the model application stage.

In this connection, it may be so designed that the measurement device is connected to the machine learning apparatus 100 and the machine learning apparatus 100 receives the measurement data directly from the measurement device. Alternatively, it may be so designed that the measurement device is connected to the machine learning apparatus 100 over a local network or a wide area network, and the machine learning apparatus 100 receives the measurement data over the network. Yet alternatively, it may be so designed that the measurement device once sends the measurement data to another information processing apparatus, and the machine learning apparatus 100 collects the measurement data from the other information processing apparatus. Yet alternatively, it may be so designed that the measurement data is stored in a storage medium and the machine learning apparatus 100 reads the measurement data from the storage medium. In addition, the label that is given to the measurement data may be supplied to the machine learning apparatus 100 by a user. In addition, the machine learning apparatus 100 may receive the label together with the measurement data from another information processing apparatus or may read the label together with the measurement data from the storage medium.

The filter storage unit 123 holds a filter used for preprocessing measurement data. The filter may be a low-pass filter to remove high-frequency noise. The filter storage unit 123 may hold information on a cutoff frequency and filter order, or may hold information on a filter coefficient for an FIR filter or IIR filter. In addition, the filter storage unit 123 may hold definitions about a plurality of filters, and the preprocessing units 131 and 133 may select one of the plurality of filters. In addition, it may be so designed that the filter update unit 135 creates a new filter and stores it in the filter storage unit 123.

The training data storage unit 124 holds training data used for training a model. The training data is obtained by preprocessing the measurement data stored in the measurement data storage unit 121. The preprocessing may include noise removal using a low-pass filter. The preprocessing may also include adjusting the time duration and amplitude of a time-series signal. Without substantially performing the preprocessing, the measurement data itself may be used as training data.

The model storage unit 125 holds a model trained using the training data. The model is a classifier to classify input data into a plurality of classes. For example, the model determines whether the input data is normal or abnormal. The model is a neural network, a support vector machine, a regression analysis model, a random forest, a k-nearest neighbor model, or another.

The prediction result storage unit 126 holds prediction results obtained by the model stored in the model storage unit 125 from the measurement data stored in the measurement data storage unit 122. A prediction result indicates whether the measurement data is normal or abnormal, for example. The prediction result is correct if the prediction result agrees with the label or is incorrect if the prediction result does not agree with the label. On the basis of the prediction results, it is possible to calculate prediction accuracy as an evaluation value. The prediction accuracy is defined by accuracy representing the ratio of the number of input samples with correct prediction results among a predetermined number of recent input samples. In this connection, an index other than the accuracy may be used as the prediction accuracy.

The preprocessing unit 131 preprocesses the measurement data stored for training in the measurement data storage unit 121 to thereby generate preprocessed training data. The preprocessing unit 131 stores the training data in the training data storage unit 124 and supplies the training data to the model training unit 132. As the preprocessing, the preprocessing unit 131 may use a filter stored in the filter storage unit 123. For example, the preprocessing unit 131 removes high-frequency noise from the measurement data using a low-pass filter. The filter used by the preprocessing unit 131 may be determined by trial and error by a user so as to improve the prediction accuracy of the model. In this connection, an appropriate preprocessing filter may be searched for through machine learning. In addition, as the preprocessing, the preprocessing unit 131 may adjust the time duration and amplitude of a time-series signal.

The model training unit 132 creates a model through machine learning using the training data preprocessed by the preprocessing unit 131 and stores the created model in the model storage unit 125. For example, the model training unit 132 creates a k-nearest neighbor model having a plurality of normal samples that are the training data. For example, the k-nearest neighbor model calculates the distance (minimum distance) between an input sample and a normal sample most similar to the input sample, and determines that the input sample is normal if the distance is less than or equal to a threshold and that the input sample is abnormal if the distance exceeds the threshold.

When new measurement data is stored in the measurement data storage unit 122, the preprocessing unit 133 preprocesses the new measurement data to generate preprocessed input data. The preprocessing unit 133 supplies the input data to the prediction unit 134. As the preprocessing, the preprocessing unit 133 may use a filter stored in the filter storage unit 123. In principal, the filter used by the preprocessing unit 133 is the same as used by the preprocessing unit 131 at the model training stage. However, the filter update unit 135 may change the filter to another that is different from the model training stage. In addition, as the preprocessing, the preprocessing unit 133 may adjust the time duration and amplitude of a time-series signal. The time duration and amplitude are adjusted in the same way as in the model training stage. In addition, in response to a request from the filter update unit 135, the preprocessing unit 133 supplies filtered input data and unfiltered input data to the filter update unit 135.

The prediction unit 134 enters the input data preprocessed by the preprocessing unit 133 into a model stored in the model storage unit 125 to predict a class to which the input data belongs. For example, the prediction unit 134 predicts whether the input data is normal or abnormal. The prediction unit 134 generates a prediction result indicating a class to which the input data belongs and stores the prediction result in the prediction result storage unit 126. The prediction unit 134 may additionally display the prediction result on the display device 111 or sends it to another information processing apparatus.

The filter update unit 135 updates the filter that is used by the preprocessing unit 133 when the prediction accuracy of the model decreases after the application starts. More specifically, the filter update unit 135 reads the prediction result output from the prediction unit 134 from the prediction result storage unit 126 and compares the prediction result with the label given to the measurement data. The filter update unit 135 determines that the prediction result is correct if the label and the prediction result indicate the same classification class and that the prediction result is incorrect if the label and the prediction result indicate different classification classes. The filter update unit 135 calculates prediction accuracy such as accuracy on the basis of comparison results for a predetermined number of recent input samples. In the case where the latest prediction accuracy falls below a threshold, the filter update unit 135 determines to update the preprocessing filter. The threshold for the prediction accuracy may be set to a fixed value in advance or may be determined on the basis of the prediction accuracy obtained at the time of training the model.

When updating the filter, the filter update unit 135 obtains recently-filtered input data from the preprocessing unit 133 and specifies input data that has caused the decrease in the prediction accuracy. For example, the input data that has caused the decrease in the prediction accuracy is input data whose distance to training data exceeds a threshold. For example, with reference to the training data stored in the training data storage unit 124, the filter update unit 135 may specify the cause of the decrease in the prediction accuracy. Alternatively, the filter update unit 135 may specify an input sample erroneously determined to be abnormal among the input samples associated with a label indicating normal, as the cause of the decrease in the prediction accuracy.

When having specified input data that has caused the decrease in the prediction accuracy, the filter update unit 135 obtains unfiltered input data, which has not passed through a filter, corresponding to the cause from the preprocessing unit 133. The filter update unit 135 creates a filter with changed parameters, enters the input data to the created filter, and calculates the distance between the filtered input data and the training data. For example, the filter update unit 135 creates a low-pass filter with a changed cutoff frequency and changed filter order and enters the input data into the created low-pass filter. The filter update unit 135 adjusts the parameters of the filter so as to yield a small distance. In this way, the filter update unit 135 updates the filter that is used by the preprocessing unit 133. The filter update unit 135 may save the created filter in the filter storage unit 123.

In this connection, the filter update unit 135 may be designed to determine whether the distance between input data after the filter optimization and the training data is less than or equal to a predetermined threshold and to determine that the filter optimization has failed if the distance exceeds the threshold. This is because, in the case where the tendency of noise contained in measurement data is greatly different from the model training stage, there is a possibility that the prediction accuracy of the model is not sufficiently recovered by only performing the filter optimization. In the case of failure, the retraining of the model using the latest measurement data is preferable. For example, in the case where the distance between the input data after the filter optimization and the training data exceeds the threshold, the filter update unit 135 may output a warning to promote the retraining of the model. The threshold may be the same as used by the k-nearest neighbor model in the classification into normal and abnormal. The warning may be displayed on the display device 111 or sent to another information processing apparatus.

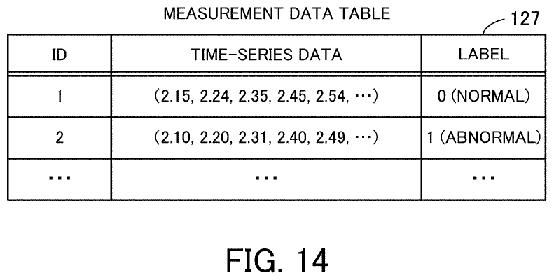

FIG. 14 illustrates an example of a measurement data table. A measurement data table 127 is stored in the measurement data storage unit 122. The measurement data storage unit 121 may hold the same table as the measurement data table 127. The measurement data table 127 includes the following items: ID, time-series data, and label. An ID identifies a sample of time-series data. The time-series data is primary data whose signal level varies along a time axis, such as electrocardiogram data or walking data. The signal level of the time-series data is measured at a predetermined sampling rate. A label indicates a correct classification class to which the time-series data belongs. For example, the label indicates normal or abnormal.

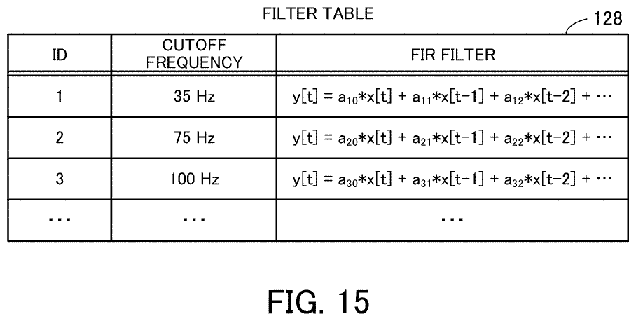

FIG. 15 illustrates an example of a filter table. The filter table 128 is stored in the filter storage unit 123. The filter table 128 has the following items: ID, cutoff frequency, and FIR filter. An ID identifies a low-pass filter. A cutoff frequency indicates a boundary between frequencies for passing and frequencies for cutoff. An FIR filter acting as a low-pass filter is defined by a linear equation including a filter coefficient by which the latest input signal and a predetermined number of past input signals are each multiplied. In this connection, the low-pass filter may be implemented by using another filter such as an IIR filter. The cutoff frequency is one of the parameters of the low-pass filter. The parameters of the low-pass filter may include a filter order. In addition, the parameters of the low-pass filter may include an amplitude indicating an attenuation ratio around the cutoff frequency. In addition, the filter coefficient may be set as one of adjustable parameters of the low-pass filter.

The following describes how the machine learning apparatus 100 operates. In the following description, assume the case of determining whether electrocardiogram data is normal or abnormal with the k-nearest neighbor algorithm.

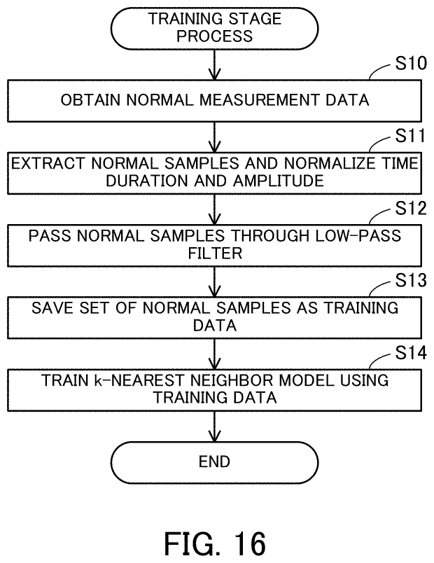

FIG. 16 is a flowchart illustrating an example of a training stage process. (S10) The preprocessing unit 131 obtains normal measurement data. Abnormal measurement data does not need to be obtained or a label does not need to be explicitly given to the measurement data.

(S11) The preprocessing unit 131 extracts a plurality of normal samples for a predetermined number of periods from the measurement data and normalizes the time duration and amplitude of each normal sample.

(S12) The preprocessing unit 131 passes each of the plurality of normal samples through a low-pass filter. The parameters set in the low-pass filter, such as a cutoff frequency and a filter order, are specified by a user. In this connection, it may be so designed as not to pass the normal samples through the low-pass filter. Alternatively, the low-pass filter may substantially be deactivated by adjusting the parameters of the low-pass filter, for example, by setting a sufficiently high cutoff frequency.

(S13) The preprocessing unit 131 generates a set of normal samples having been subjected to the preprocessing including steps S11 and S12, as training data, and saves the training data in the training data storage unit 124.

(S14) The model training unit 132 trains a k-nearest neighbor model using the training data. The k-nearest neighbor model trained here is a nearest neighbor model that obtains the minimum distance among the distances between an input sample and each of the plurality of normal samples, and determines that the input sample is normal if the minimum distance is less than or equal to a threshold and that the input sample is abnormal if the minimum distance exceeds the threshold. The threshold may be specified by the user. The model training unit 132 stores the k-nearest neighbor model in the model storage unit 125.

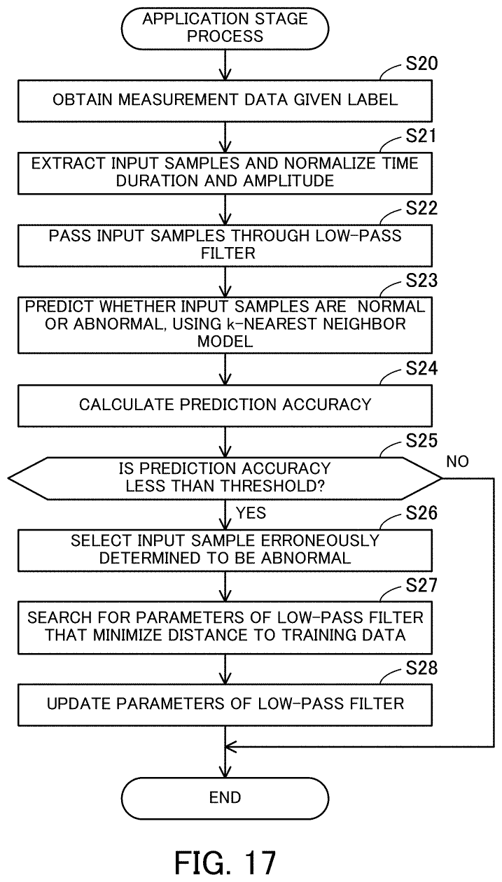

FIG. 17 is a flowchart illustrating an example of an application stage process. (S20) The preprocessing unit 133 obtains measurement data obtained after the model training. This measurement data is given a label indicating normal or abnormal. For example, the label is fed back for the measurement data by a specialist such as a medical worker.

(S21) The preprocessing unit 133 extracts a plurality of input samples for a predetermined number of periods from the measurement data, and normalizes the time duration and amplitude of each input sample.

(S22) The preprocessing unit 133 passes each of the plurality of input samples through a low-pass filter. In principal, the parameters set in the low-pass filter, such as a cutoff frequency and filter order, are the same as used in the model training. In this connection, in the case where the parameters are changed after the model training as described later, the newest parameters are used.

(S23) The prediction unit 134 reads a k-nearest neighbor model from the model storage unit 125. The prediction unit 134 enters the input samples subjected to the preprocessing including steps S21 and S22 into the k-nearest neighbor model to predict whether each input sample is normal or abnormal. The prediction unit 134 stores the prediction results indicating normal or abnormal in the prediction result storage unit 126. The prediction unit 134 may display the prediction results on the display device 111 or may send the prediction results to another information processing apparatus.

(S24) The filter update unit 135 calculates the latest prediction accuracy of the k-nearest neighbor model. For example, the filter update unit 135 compares, with respect to a plurality of recent input samples, the prediction result of each input sample with the label, and calculates accuracy representing the ratio of the number of input samples whose prediction results agree with the label. For example, the accuracy is used as an index of the prediction accuracy.

(S25) The filter update unit 135 determines whether the prediction accuracy is less than a threshold. The threshold may be specified by the user at the model training stage or after the start of the model application. Alternatively, the threshold may automatically be determined on the basis of the prediction accuracy of the k-nearest neighbor model obtained at the training time. The process proceeds to step S26 if the prediction accuracy is less than the threshold; otherwise the processing of the obtained measurement data is completed.

(S26) The filter update unit 135 selects, as a cause of the decrease in the prediction accuracy, an input sample that has erroneously been determined to be abnormal by the k-nearest neighbor model from the input samples given the label indicating normal. The selected input sample is a normal input sample and its distance to the training data (the minimum distance among the distances to a plurality of normal samples) exceeds the threshold. This distance is calculated between the input sample having passed through the low-pass filter and the training data. In this connection, using a threshold different from that for the k-nearest neighbor model, an input sample whose distance to the training data exceeds the threshold may be selected from the normal input samples.

(S27) The filter update unit 135 uses an unfiltered input sample, which has not passed through the low-pass filter, corresponding to the input sample selected at step S26 to search for the parameters of the low-pass filter. The filter update unit 135 enters the unfiltered input sample into a low-pass filter having changed parameters such as a changed cutoff frequency and changed filter order, and calculates the distance between the input sample having passed through the low-pass filter and the training data. The filter update unit 135 adjusts the parameters of the low-pass filter so as to minimize the distance. In this connection, to search for parameters that yield the minimum distance, a simple search method of trying some parameters or an optimization algorithm such as the gradient descent may be used.

(S28) The filter update unit 135 updates the parameters of the low-pass filter. The updated parameters yield the minimum distance at step S27. The updated parameters are used for subsequently obtained measurement data.

As described above, the machine learning apparatus 100 of the second embodiment trains a model using preprocessed training data at the model training stage, and enters preprocessed input data into the model at the model application stage. This makes it possible to train the model with high prediction accuracy from measurement data containing noise and to keep the prediction accuracy at the model application stage. Thus, for example, it is possible to perform classification of input data, such as classification into normal and abnormal, with high accuracy.

When the tendency of noise changes at a later time due to changes in the hardware characteristics and usage environment of a measurement device, the parameters of the preprocessing are updated. Therefore, it is expected that the influence of the change on preprocessed input data is suppressed and the prediction accuracy is recovered up to the level obtained at the model training stage without retraining the model. The unnecessity of retraining the model avoids an increase in cost such as the computational complexity and training time of the machine learning. In addition, the training data is stored at the model training stage, and the parameters of the preprocessing are automatically adjusted so that the tendency of preprocessed input data gets closer to that of the training data used at the model training stage. Thus, appropriate filtering is performed without excessive filtering or insufficient removal of noise, which increases a possibility of improving the prediction accuracy.

According to one aspect, it is possible to minimize the necessity of retraining a model due to a change in the tendency of data.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. A computer-implemented data processing method comprising:
generating training data by performing a process, based on a parameter, on first measurement data;
training a machine learning model by using the training data;
generating first data by performing the process on second measurement data;
generating a first prediction result by entering the first data into the machine learning model, and calculating prediction accuracy based on a label associated with the second measurement data and the first prediction result; and
changing the parameter of the process in accordance with a comparison between the training data and the first data in response to the predication accuracy being less than a threshold.

2. The data processing method according to claim 1, further comprising:
generating second data by performing the process based on the changed parameter on third measurement data and generating a second prediction result by entering the second data into the machine learning model.

3. The data processing method according to claim 1, wherein:
the parameter includes a cutoff frequency; and
the process includes low-frequency filtering to reduce high-frequency components higher than the cutoff frequency.

4. The data processing method according to claim 1, wherein the machine learning model calculates a distance between the first data and the training data and classifies, based on the distance, the first data into normal or abnormal.

5. The data processing method according to claim 1,
wherein the changing of the parameter includes calculating a distance between the training data and the first data and adjusting the parameter so as to reduce the distance.

6. A data processing apparatus comprising:
a memory that holds first measurement data, training data, a machine learning model, second measurement data, and a label associated with the second measurement data; and
a processor coupled to the memory, the processor being configured to
generate the training data by performing a process based on a parameter, on the first measurement data,
train the machine learning model by using the training data,
generate first data by performing the process on the second measurement data,
generate a first prediction result by entering the first data into the machine learning model, and calculate prediction accuracy based on the label and the first prediction result, and
change the parameter of the process in accordance with a comparison between the training data and the first data in response to the prediction accuracy being less than a threshold.

7. A non-transitory computer-readable storage medium storing a program executable by one or more computers, the program comprising:
an instruction for generating training data by performing a process, based on a parameter, on first measurement data;
an instruction for training a machine learning model by using the training data;
an instruction for generating first data by performing the process on second measurement data;
an instruction for generating a first prediction result by entering the first data into the machine learning model, and calculating prediction accuracy based on a label associated with the second measurement data and the first prediction result; and
an instruction for changing the parameter of the process in accordance with a comparison between the training data and the first data in response to the predication accuracy being less than a threshold.
